(19) United States
(12) Patent Application Publication
(10) Pub. No.: US 2022/0185781 A1
(43) Pub. Date: Jun. 16, 2022

(54) BULLEYACONITINE D CRYSTAL AND PREPARATION METHOD THEREFOR AND APPLICATION THEREOF

(71) Applicant: YUNNAN HAOPY PHARMACEUTICALS LTD, Kunming (CN)

(72) Inventors: Qiongfen WU, Kunming (CN); Biao LI, Kunming (CN)

(21) Appl. No.: 17/438,914

(22) PCT Filed: Feb. 21, 2020
(86) PCT No.: PCT/CN2020/076155

§ 371 Date: Sep. 13, 2021

(30) Foreign Application Priority Data
Mar. 15, 2019 (CN) 201910198109.3

(51) Int. Cl. C07D 221/22

(52) U.S. Cl. CPC C07D 221/22; C07B 2200/13

(57) ABSTRACT

Disclosed in the present invention are a bulleyaconitine D crystal and a preparation method therefor. FIG. 1 shows an X-ray powder diffraction spectrum of the crystal according to the present invention, the spectrum being measured with Cu—K alpha ray. The bulleyaconitine D crystal is prepared by an anti-solvent process with isopropanol, anisole, 1,4-dioxane or methylbenzene acting as a positive solvent and n-heptane as a negative solvent. The preparation process is simple, and the prepared crystal has a high purity. Upon characterization via XRD, DSC, TGA and ¹HNMR, the crystal is determined as D crystal type. Stability test shows that the prepared bulleyaconitine crystal is well stable to light, damp and heat.

This application claims the priority of Chinese Patent Application No. 201910198109.3, filed with the China National Intellectual Property Administration on Mar. 15, 2019, and titled with “BULLEYACONITINE D CRYSTAL AND PREPARATION METHOD THEREFOR AND APPLICATION THEREOF”, and the disclosures of which are hereby incorporated by reference.

### FIELD

The present disclosure relates to the field of medicinal chemistry, specifically to a crystalline form D of bulleyaconitine A and preparation method therefor and application thereof.

### BACKGROUND

Bulleyaconitine has a chemical name of (1α,6α,14α,16β)tetrahydro-8,13,14-triol-20-ethyl-1,6,16-trimethoxy-4-methoxymethyl-8-acetoxy-14-(4′-p-methoxybenzyl)-aconitine. It is a diterpene diester alkaloid extracted and isolated from the root tuber of Aconitum georgei Comber, a plant of the genus Aconitum in the Ranunculaceae family, named Crassicauline A, and later, it was renamed Bulleyaconitine A (T2). It is a known natural compound in plant species, and its structural formula is as follows:

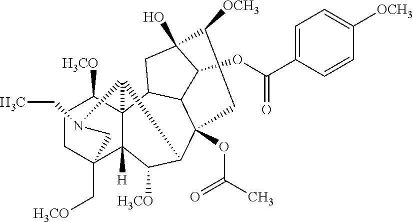

At present, bulleyaconitine A preparations are widely used clinically to treat rheumatoid arthritis (RA), osteoarthritis, myofibrositis, pain in neck and shoulder, pain in lower extremities and waist, cancerous pain and chronic pain caused by various reasons.

Polymorphism in pharmaceuticals is a common phenomenon in drug research and development, and is an important factor which influences drug quality. The same drugs with different crystalline forms vary in appearance, solubility, melting point, dissolution, and bioavailability, and may even have significant differences. Therefore, the crystalline form of the drug will affect the stability, bioavailability and therapeutic effect. Moreover, the crystalline form of a drug will also affect the quality and absorption behavior in human body of a pharmaceutical preparation of the drug, and finally affects the benefit ratio between the therapeutic effect and side effect of the preparation in human body. With the in-depth research of bulleyaconitine A, the research on the crystalline form and physicochemical properties of bulleyaconitine A is of great significance to the evaluation of the drug efficacy, quality, and safety of bulleyaconitine A. The Chinese patent with application number 201710423005.9 discloses that bulleyaconitine A is dissolved with a C1-4 organic solvent, then the obtained bulleyaconitine A solution is added dropwise to water, stirring while adding, and after the addition, suction filtration is performed and the filter cake is dried to obtain the amorphous bulleyaconitine A. So far, there is no relevant report on crystalline bulleyaconitine A.

### SUMMARY

In view of this, the purpose of the present disclosure is to provide a new crystalline form of bulleyaconitine A and a preparation method thereof.

An object of the present disclosure is to research, discover and provide the crystalline form D of bulleyaconitine A by crystallographic methods.

In the present disclosure, X-ray powder diffraction (XRPD), which is internationally acknowledged, is adopted to study and characterize the crystalline form of bulleyaconitine A. Measurement conditions and methods: Cu/K-alpha1 (target), 45 KV-40 mA (working voltage and current), 2θ=3-40 (scanning range), scanning time per step (s) is 17.8-46.7, scanning step size (2θ) is 0.0167-0.0263, λ=1.54 Å.

The substantially pure crystalline form D of bulleyaconitine A provided by the present disclosure has an X-ray powder diffraction spectrum as shown in FIG. 1, and its X-ray powder diffraction spectrum shows obvious characteristic absorption peaks at 2θ values of 7.3±0.2, 9.3±0.2, 11.8±0.2, 12.3±0.2, 13.8±0.2, 14.5±0.2, 15.7±0.2, 18.7±0.2, 21.8±0.2, 22.9±0.2, and 29.8±0.2.

The present disclosure also adopts thermogravimetric analysis to study and characterize the crystalline form D of bulleyaconitine A. The detection conditions are: as the temperature rise gradient, increasing temperature from room temperature to 400° C. at a rate of 10° C./min, with nitrogen as the protective gas.

The substantially pure crystalline form D of bulleyaconitine A provided by the present disclosure has a thermogravimetric analysis graph as shown in FIG. 2, and it has the following characteristics: when the temperature rises to 150° C., the sample has a weight loss of 1.2%.

The present disclosure also adopts differential scanning calorimetry to study and characterize the crystalline form D of bulleyaconitine A. The detection method is: as the temperature rise gradient, increasing temperature from 25° C. to 280° C. at a rate of 10° C./min, with nitrogen as the protective gas.

The substantially pure crystalline form D of bulleyaconitine A provided by the present disclosure has a differential scanning calorimetry graph as shown in FIG. 2, and it has the following characteristics: the endothermic peak is 170-175° C.

It is worth noting that among the X-ray powder diffraction spectra of the above-mentioned crystalline form, the characteristic peaks of the X-ray powder diffraction spectrum may have slight differences between one machine and another machine and between one sample and another sample. The value may differ by about 1 unit, or by about 0.8 unit, or by about 0.5 unit, or by about 0.3 unit, or by about 0.1 unit, so the value given should not be regarded as absolute. Similarly, the values given in the differential scanning calorimetry graphs of the above-mentioned crystalline forms should not be regarded as absolute either.

The crystalline form can also be characterized by other analytical techniques known in the art, such as hydrogen nuclear magnetic resonance spectrum (¹HNMR).

The substantially pure crystalline form D of bulleyaconitine A provided by the present disclosure has a hydrogen nuclear magnetic resonance spectrum as shown in FIG. 3.

The present disclosure also provides a preparation method of the crystalline form D of bulleyaconitine A with high purity and no residual solvent.

The preparation method of the crystalline form D of bulleyaconitine A provided by the present disclosure comprises adding a positive solvent to a sample of bulleyaconitine A, stirring to dissolve it, adding an anti-solvent during the stirring process, precipitating a solid after standing or cooling, separating the solid by centrifugation, wherein the positive solvent is isopropanol, anisole, 1,4-dioxane or toluene, and the anti-solvent is n-heptane.

Preferably, the stirring rate when adding the anti-solvent is no less than 250 r/min.

Preferably, the volume ratio of the positive solvent to the anti-solvent is 10:1-1:10.

Preferably, the cooling is cooling from room temperature to −20° C. or any temperature point in between.

The crystalline form D of bulleyaconitine A obtained by the preparation method of the present disclosure has a crystalline form content of more than 99%, high purity, consistent X-ray powder diffraction spectrum characteristics and DSC characteristics, stable properties, and good stability to light, humidity and heat.

The present disclosure also provides use of the crystalline form D of bulleyaconitine A in the manufacture of a medicament for the prevention and/or treatment of rheumatoid arthritis (RA), osteoarthritis, myofibrositis, pain in neck and shoulder, pain in lower extremities and waist, or cancerous pain.

It can be known from the above technical solutions that the present disclosure discloses a crystalline form D of bulleyaconitine A and a preparation method thereof. The X-ray powder diffraction spectrum of the crystalline form of the present disclosure measured by Cu-Kα ray is shown in FIG. 1. The crystalline form D of bulleyaconitine A is prepared by an anti-solvent process with isopropanol, anisole, 1,4-dioxane or toluene as a positive solvent and n-heptane as an anti-solvent. The preparation process is simple, and the prepared crystalline form has a high purity and is characterized by XRD, DSC, TGA, and ¹HNMR to be determined as crystalline form D. The obtained crystalline form D of bulleyaconitine A is an anhydrous crystalline form, and the stability test shows that the crystal has good stability to light, humidity and heat.

### BRIEF DESCRIPTION OF DRAWINGS

In order to more clearly illustrate the technical solutions in the examples of the present disclosure or in the prior art, the drawings used in the examples or the prior art will be briefly introduced below.

FIG. 1 XRPD graph of the crystalline form D of bulleyaconitine A;

FIG. 2 TGA/DSC graph of the crystalline form D of bulleyaconitine A;

FIG. 3 ¹HNMR spectrum of the crystalline form D of bulleyaconitine A.

### DETAILED DESCRIPTION

Hereinafter, the technical solutions in embodiments of the present disclosure will be described clearly and completely in conjunction with examples of the present disclosure. It is apparent that the described examples are merely part of the present disclosure rather than all. Based on the examples in the present disclosure, all other examples obtained by those of ordinary skill in the art without creative work are within the scope of the present disclosure.

The present disclosure will be illustrated in detail in combination with specific examples below in order to further understand the present disclosure. In the following examples, unless otherwise specified, the test method is usually implemented in accordance with conventional conditions or conditions recommended by the manufacturer.

### Test Parameters

The XRPD patterns were collected on PANalytacal Empyrean and X' Pert3 X-ray powder diffraction analyzers. The scanning parameters are shown in Table 1.

TABLE 1
XRPD test parameters

| | Parameters | | | |
|---|---|---|---|---|
| Instrument | Reflection mode | | | Transmission mode |
| model | Empyrean | X′ Pert3 | X′ Pert3 | X′ Pert3 |
| X-ray | Cu, kα, Kα1 (Å): 1.540598; Kα2 (Å): 1.544426 | | | |
| | Kα2/Kα1 intensity ratio: 0.50 | | | |
| X-ray tube | 45 kV, 40 mA | | | |
| setting | | | | |
| Divergence slit | Automatic | Fixed ⅛° | Fixed ⅙° | Fixed ½° |

| Scanning mode | Continuous | | |
|---|---|---|---|
| Scanning range | 3~40 | | |
| (°2Theta) | | | |
| Scanning time | 17.8 | 46.7 | 33.02 |
| per step (s) | | | |
| Scanning step | 0.0167 | 0.0263 | 0.0167 |
| size (°2Theta) | | | |
| Test time | 5 min 30 s | 5 min 4 s | 10 min 11 s |

### Thermogravimetric Analysis (TGA) and Differential Scanning Calorimetry (DSC)

TGA and DSC graphs were collected on TA Q5000 TGA/TA Discovery TGA5500 thermogravimetric analyzer and TA Q2000 DSC/TA Discovery DSC2500 differential scanning calorimeter, respectively. Table 2 lists the test parameters.

TABLE 2
TGA and DSC test parameters

| Parameters | TGA | DSC |
|---|---|---|
| Method | Linear heating | Linear heating |
| Sample pan | Aluminum pan, open | Aluminum pan, gland |
| Temperature range | Room temperature- | 25° C.- End |
| | End temperature set | temperature set |
| Scanning rate | 10 | 10 |
| (° C./min) | | |
| Protective gas | Nitrogen | Nitrogen |

### Liquid NMR

The liquid NMR spectra were collected on Bruker 400M NMR spectrometer, with DMSO-d6 as the solvent.

### Example 1. Preparation and Identification of Crystalline Form D or Bulleyaconitine A

150 mg of bulleyaconitine A was weighed out and placed in a beaker. Then 5 ml of isopropanol was added at room temperature and dissolved by stirring. When the rotational speed was 500 r/min, 5 ml of n-heptane was added while stirring. After adding n-heptane, it was allowed to stand at room temperature and then centrifuged to obtain a solid. The solid was subjected to XRPD, TGA/DSC and ¹HNMR tests.

The XRPD results show that there are obvious characteristic absorption peaks at the diffraction angle (2θ angle) of 7.3±0.2, 9.8±0.2, 11.9±0.2, 12.4±0.2, 14.2±0.2, 14.8±0.2, 15.7±0.2, 18.7±0.2, 22.1±0.2, 22.8±0.2, and 29.6±0.2. The TGA/DSC results show that when the temperature rises to 150° C., the weight loss is 1.2%, and the DSC graph shows a sharp endothermic peak at 171.9° C. (initial temperature), which may be caused by melting. Combined with the TGA weight loss, it is speculated that the thermal signal appearing after 200° C. on the DSC graph may be caused by the decomposition of the sample. ¹HNMR results show that there is no obvious solvent residue in the sample.

It was identified as crystalline form D, anhydrous form.

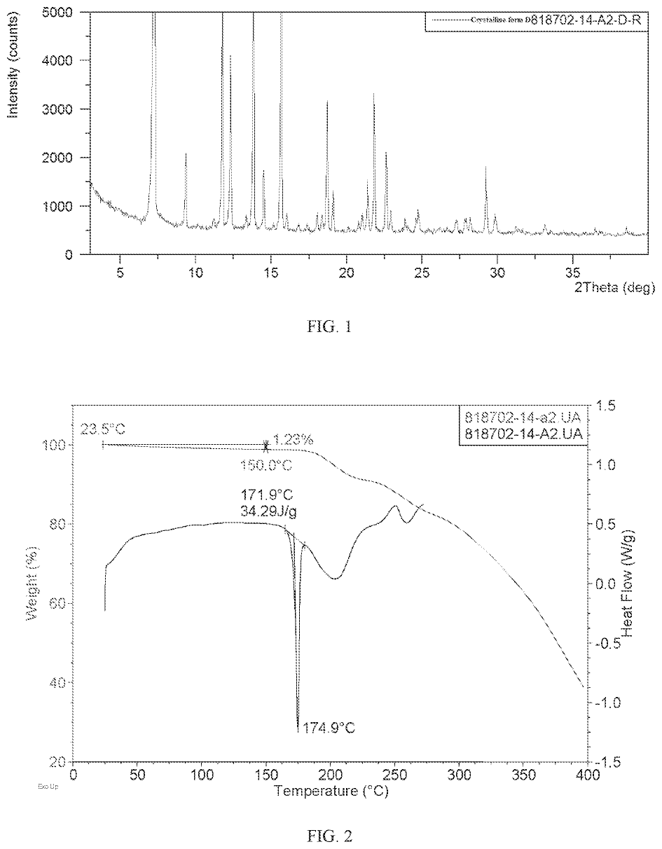

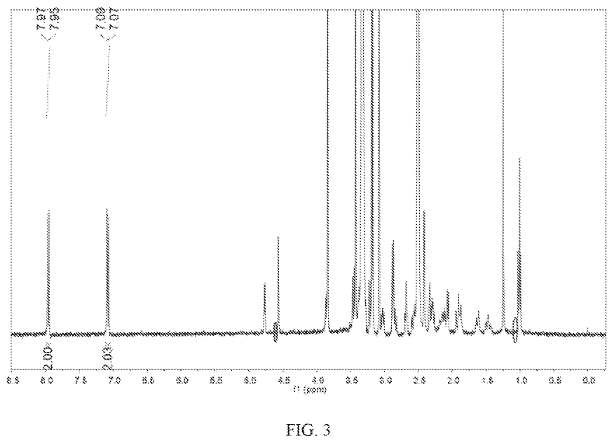

The graphs are shown in FIG. 1 X-ray powder diffraction pattern of the crystalline form D of bulleyaconitine A, FIG. 2 TGA/DSC analysis graph of the crystalline form D of bulleyaconitine A, and FIG. 3 ¹HNMR spectrum of the crystalline form D of bulleyaconitine A.

### Example 2: Preparation of Crystalline Form D of Bulleyaconitine A

150 mg of bulleyaconitine A was weighed out and placed in a beaker. Then 5 ml of isopropanol was added at room temperature and dissolved by stirring. When the rotational speed was 250 r/min, 0.5 ml of n-heptane was added while stirring. After adding n-heptane, it was allowed to stand at −20° C. and then centrifuged to separate and obtain a solid. The solid was subjected to XRPD and DSC tests. The XRPD results are consistent with the results in FIG. 1, and the endothermic peak of DSC is 170° C.

### Example 3: Preparation of Crystalline Form D of Bulleyaconitine A

150 mg of bulleyaconitine A was weighed out and placed in a beaker. Then 5 ml of isopropanol was added at room temperature and dissolved by stirring. When the rotational speed was 750 r/min, 50 ml of n-heptane was added while stirring. After adding n-heptane, it was allowed to stand at 10° C. and then centrifuged to separate and obtain a solid. The solid was subjected to XRPD and DSC tests. The XRPD results are consistent with the results in FIG. 1, and the endothermic peak of DSC is 170.6° C.

### Example 4: Preparation of Crystalline Form D of Bulleyaconitine A

150 mg of bulleyaconitine A was weighed out and placed in a beaker. Then 5 ml of isopropanol was added at room temperature and dissolved by stirring. When the rotational speed was 1000 r/min, 25 ml of n-heptane was added while stirring. After adding n-heptane, it was allowed to stand at 0° C. and then centrifuged to separate and obtain a solid. The solid was subjected to XRPD and DSC tests. The XRPD results are consistent with the results in FIG. 1, and the endothermic peak of DSC is 175° C.

### Example 5: Preparation of Crystalline Form D of Bulleyaconitine A

150 mg of bulleyaconitine A was weighed out and placed in a beaker. Then 5 ml of anisole was added at room temperature and dissolved by stirring. When the rotational speed was 500 r/min, 15 ml of n-heptane was added while stirring. After adding n-heptane, it was allowed to stand at room temperature and then centrifuged to separate and obtain a solid. The solid was subjected to XRPD and DSC tests. The XRPD results are consistent with the results in FIG. 1, and the endothermic peak of DSC is 174.8° C.

### Example 6: Preparation of Crystalline Form D of Bulleyaconitine A

150 mg of bulleyaconitine A was weighed out and placed in a beaker. Then 5 ml of anisole was added at room temperature and dissolved by stirring. When the rotational speed was 250 r/min, 0.5 ml of n-heptane was added while stirring. After adding n-heptane, it was allowed to stand at −20° C. and then centrifuged to separate and obtain a solid. The solid was subjected to XRPD and DSC tests. The XRPD results are consistent with the results in FIG. 1, and the endothermic peak of DSC is 173.5° C.

### Example 7: Preparation of Crystalline Form D of Bulleyaconitine A

150 mg of bulleyaconitine A was weighed out and placed in a beaker. Then 5 ml of anisole was added at room temperature and dissolved by stirring. When the rotational speed was 750 r/min, 50 ml of n-heptane was added while stirring. After adding n-heptane, it was allowed to stand at 10° C. and then centrifuged to separate and obtain a solid. The solid was subjected to XRPD and DSC tests. The XRPD results are consistent with the results in FIG. 1, and the endothermic peak of DSC is 171.6° C.

### Example 8: Preparation of Crystalline Form D of Bulleyaconitine A

150 mg of bulleyaconitine A was weighed out and placed in a beaker. Then 5 ml of anisole was added at room temperature and dissolved by stirring. When the rotational speed was 1000 r/min, 25 ml of n-heptane was added while stirring. After adding n-heptane, it was allowed to stand at 0° C. and then centrifuged to separate and obtain a solid. The solid was subjected to XRPD and DSC tests. The XRPD results are consistent with the results in FIG. 1, and the endothermic peak of DSC is 172.4° C.

### Example 9: Preparation of Crystalline Form D of Bulleyaconitine A

150 mg of bulleyaconitine A was weighed out and placed in a beaker. Then 5 ml of 1,4-dioxane was added at room temperature and dissolved by stirring. When the rotational speed was 250 r/min, 0.5 ml of n-heptane was added while stirring. After adding n-heptane, it was allowed to stand at −20° C. and then centrifuged to separate and obtain a solid. The solid was subjected to XRPD and DSC tests. The XRPD results are consistent with the results in FIG. 1, and the endothermic peak of DSC is 171.8° C.

### Example 10: Preparation of Crystalline Form D of Bulleyaconitine A

150 mg of bulleyaconitine A was weighed out and placed in a beaker. Then 5 ml of 1,4-dioxane was added at room temperature and dissolved by stirring. When the rotational speed was 250 r/min, 25 ml of n-heptane was added while stirring. After adding n-heptane, it was allowed to stand at room temperature and then centrifuged to separate and obtain a solid. The solid was subjected to XRPD and DSC tests. The XRPD results are consistent with the results in FIG. 1, and the endothermic peak of DSC is 172.6° C.

### Example 11: Preparation of Crystalline Form D of Bulleyaconitine A

150 mg of bulleyaconitine A was weighed out and placed in a beaker. Then 5 ml of 1,4-dioxane was added at room temperature and dissolved by stirring. When the rotational speed was 750 r/min, 50 ml of n-heptane was added while stirring. After adding n-heptane, it was allowed to stand at 10° C. and then centrifuged to separate and obtain a solid. The solid was subjected to XRPD and DSC tests. The XRPD results are consistent with the results in FIG. 1, and the endothermic peak of DSC is 173.4° C.

### Example 12: Preparation of Crystalline Form D of Bulleyaconitine A

150 mg of bulleyaconitine A was weighed out and placed in a beaker. Then 5 ml of 1,4-dioxane was added at room temperature and dissolved by stirring. When the rotational speed was 1000 r/min, 25 ml of n-heptane was added while stirring. After adding n-heptane, it was allowed to stand at 0° C. and then centrifuged to separate and obtain a solid. The solid was subjected to XRPD and DSC tests. The XRPD results are consistent with the results in FIG. 1, and the endothermic peak of DSC is 174.7° C.

### Example 13: Preparation of Crystalline Form D of Bulleyaconitine A

150 mg of bulleyaconitine A was weighed out and placed in a beaker. Then 5 ml of toluene was added at room temperature and dissolved by stirring. When the rotational speed was 250 r/min, 0.5 ml of n-heptane was added while stirring. After adding n-heptane, it was allowed to stand at −20° C. and then centrifuged to separate and obtain a solid. The solid was subjected to XRPD and DSC tests. The XRPD results are consistent with the results in FIG. 1, and the endothermic peak of DSC is 175° C.

### Example 14: Preparation of Crystalline Form D of Bulleyaconitine A

150 mg of bulleyaconitine A was weighed out and placed in a beaker. Then 5 ml of toluene was added at room temperature and dissolved by stirring. When the rotational speed was 750 r/min, 35 ml of n-heptane was added while stirring. After adding n-heptane, it was allowed to stand at room temperature and then centrifuged to separate and obtain a solid. The solid was subjected to XRPD and DSC tests. The XRPD results are consistent with the results in FIG. 1, and the endothermic peak of DSC is 170.2° C.

### Example 15: Preparation of Crystalline Form D of Bulleyaconitine A

150 mg of bulleyaconitine A was weighed out and placed in a beaker. Then 5 ml of toluene was added at room temperature and dissolved by stirring. When the rotational speed was 750 r/min, 50 ml of n-heptane was added while stirring. After adding n-heptane, it was allowed to stand at 10° C. and then centrifuged to separate and obtain a solid. The solid was subjected to XRPD and DSC tests. The XRPD results are consistent with the results in FIG. 1, and the endothermic peak of DSC is 171.2° C.

### Example 16: Preparation of Crystalline Form D of Bulleyaconitine A

150 mg of bulleyaconitine A was weighed out and placed in a beaker. Then 5 ml of toluene was added at room temperature and dissolved by stirring. When the rotational speed was 1000 r/min, 25 ml of n-heptane was added while stirring. After adding n-heptane, it was allowed to stand at 0° C. and then centrifuged to separate and obtain a solid. The solid was subjected to XRPD and DSC tests. The XRPD results are consistent with the results in FIG. 1, and the endothermic peak of DSC is 173.8° C.

### Example 17. Stability Test of Crystalline Form D of Bulleyaconitine a

In order to evaluate the solid-state stability of crystalline form D, an appropriate amount of samples was weigh out and placed in an open place at 25° C./60% RH and 40° C./75% RH for 1 week and 1 month, respectively, and placed in a sealed place at 80° C. for 24 hours. XRPD and HPLC characterization of the placed samples were performed to detect the changes of crystalline form and chemical purity.

The HPLC results are shown in Table 3 that the chemical purity of the sample has hardly changed under the selected test conditions; and the XRPD results show that the crystalline form of the sample has not changed under the selected test conditions.

TABLE 3
Summary of stability data of crystalline form D

| Crystalline form | | | | HPLC purity | | |
|---|---|---|---|---|---|---|
| (Sample No.) | Conditions | | | Area % | % of Control | Final crystalline form |
| Crystalline form D | 80° C., 24 hours | | | 99.56 | 99.9 | Crystalline form D |
| | 25° C./60% RH | 1 | week | 99.81 | 99.9 | |
| | | 1 | month | 99.29 | 100.0 | |
| | 40° C./75% RH | 1 | week | 99.34 | 100.1 | |
| | | 1 | month | 99.74 | 100.1 | |

In conclusion, the crystalline form D has good physical and chemical stability.

1. A crystalline form D of bulleyaconitine A, wherein its X-ray powder diffraction spectrum shows obvious characteristic absorption peaks at 2θ values of 7.3±0.2, 9.3±0.2, 11.8±0.2, 12.3±0.2, 13.8±0.2, 14.5±0.2, 15.7±0.2, 18.7±0.2, 21.8±0.2, 22.9±0.2, and 29.8±0.2.

2. The crystalline form D of bulleyaconitine A according to claim 1, wherein its thermogravimetric analysis graph shows a weight loss of 1.2% when heated to 150° C.

3. The crystalline form D of bulleyaconitine A according to claim 1, wherein its differential scanning calorimetry analysis graph shows an endothermic peak at 170-175° C.

4. The crystalline form D of bulleyaconitine A according to claim 1, wherein its hydrogen nuclear magnetic resonance spectrum is shown in FIG. 3.

5. A preparation method of the crystalline form D of bulleyaconitine A according to claim 1, comprising adding a positive solvent to a sample of bulleyaconitine A, stirring to dissolve it, adding an anti-solvent during the stirring process, precipitating a solid after standing or cooling, separating the solid by centrifugation, wherein the positive solvent is isopropanol, anisole, 1,4-dioxane or toluene, and the anti-solvent is n-heptane.

6. The preparation method of the crystalline form D of bulleyaconitine A according to claim 5, wherein the stirring rate when adding the anti-solvent is no less than 250 r/min.

7. The preparation method of the crystalline form D of bulleyaconitine A according to claim 5, wherein a volume ratio of the positive solvent to the anti-solvent is 10:1-1:10.

8. The preparation method of the crystalline form D of bulleyaconitine A according to claim 5, wherein the cooling is cooling from room temperature to −20° C. or any temperature point in between.

9. A method for preventing and/or treating rheumatoid arthritis, osteoarthritis, myofibrositis, pain in neck and shoulder, pain in lower extremities and waist, or cancerous pain, comprising administering a therapeutically effective amount of the crystalline form D of bulleyaconitine A according to claim 1 to a subject in need thereof.
